(19) United States
(12) Patent Application Publication
(10) Pub. No.: US 2018/0193650 A1
(43) Pub. Date: Jul. 12, 2018

(54) PAIN MANAGEMENT BASED ON CARDIOVASCULAR PARAMETERS

(71) Applicant: Boston Scientific Neuromodulation Corporation, Valencia, CA (US)

(72) Inventors: Kyle Harish Srivastava, Saint Paul, MN (US); Pramodsingh Hirasingh Thakur, Woodbury, MN (US); Elizabeth Mary Annoni, White Bear Lake, MN (US); Jianwen Gu, Valencia, CA (US); Bryan Allen Clark, Forest Lake, MN (US); Thomas Gaviao Kilmar, Sao Paulo (BR)

(21) Appl. No.: 15/867,789

(22) Filed: Jan. 11, 2018

Related U.S. Application Data
(60) Provisional application No. 62/445,053, filed on Jan. 11, 2017.

(51) Int. Cl. A61N 1/36; A61B 5/024; A61B 5/00; A61N 1/05; A61N 1/372; A61B 7/02; A61B 5/0456; A61B 5/026

(52) U.S. Cl. CPC A61N 1/36139; A61N 1/0534; A61B 5/4824; A61B 5/4836; A61B 5/686; A61N 1/0551; A61N 1/36062; A61N 1/36071; A61N 1/37211; A61B 7/023; A61B 5/0456; A61B 5/0261; A61B 2562/0219; A61B 2562/0247; A61B 5/02416

(57) ABSTRACT

This document discusses, among other things, systems and methods for managing pain in a subject. A system may include a sensor circuit configured to sense one or more physiological signals. A pain analyzer circuit may generate from the physiological signals cardiovascular parameters indicative of arterial pulsatile activity or cardiac electrical activity, and generate a pain score using at least the cardiovascular parameters. The system may include a neurostimulator that can adaptively control the delivery of pain therapy by adjusting stimulation parameters based on the pain score.

### CLAIM OF PRIORITY

This application claims the benefit of priority under 35 U.S.C. § 119(e) of U.S. Provisional Patent Application Ser. No. 62/445,053, filed on Jan. 11, 2017, which is herein incorporated by reference in its entirety.

### CROSS REFERENCE TO RELATED APPLICATIONS

This application is related to commonly assigned U.S. Provisional Patent Application Ser. No. 62/445,061, entitled “PAIN MANAGEMENT BASED ON BRAIN ACTIVITY MONITORING”, filed on Jan. 11, 2017, U.S. Provisional Patent Application Ser. No. 62/445,069, entitled “PAIN MANAGEMENT BASED ON RESPIRATION-MEDIATED HEART RATES”, filed on Jan. 11, 2017, U.S. Provisional Patent Application Ser. No. 62/445,075, entitled “PAIN MANAGEMENT BASED ON FUNCTIONAL MEASUREMENTS”, filed on Jan. 11, 2017, U.S. Provisional Patent Application Ser. No. 62/445,082, entitled “PAIN MANAGEMENT BASED ON EMOTIONAL EXPRESSION MEASUREMENTS”, filed on Jan. 11, 2017, U.S. Provisional Patent Application Ser. No. 62/445,092, entitled “PAIN MANAGEMENT BASED ON MUSCLE TENSION MEASUREMENTS”, filed on Jan. 11, 2017, U.S. Provisional Patent Application Ser. No. 62/445,095, entitled “PATIENT-SPECIFIC CALIBRATION OF PAIN QUANTIFICATION”, filed on Jan. 11, 2017, U.S. Provisional Patent Application Ser. No. 62/395,641, entitled “METHOD AND APPARATUS FOR PAIN MANAGEMENT USING HEART SOUNDS”, filed on Sep. 16, 2016, U.S. Provisional Patent Application Ser. No. 62/400,313, entitled “SYSTEMS AND METHODS FOR CLOSED-LOOP PAIN MANAGEMENT”, filed on Sep. 27, 2016, U.S. Provisional Patent Application Ser. No. 62/400,336, entitled “METHOD AND APPARATUS FOR PAIN MANAGEMENT USING OBJECTIVE PAIN MEASURE”, filed on Sep. 27, 2016, U.S. Provisional Patent Application Ser. No. 62/412,587, entitled “METHOD AND APPARATUS FOR PAIN CONTROL USING BAROREFLEX SENSITIVITY DURING POSTURE CHANGE”, filed on Oct. 25, 2016, which are incorporated by reference in their entirety.

### TECHNICAL FIELD

This document relates generally to medical systems and more particularly to systems, devices, and methods for pain management.

### BACKGROUND

Pain is one of the most common and among the most personally compelling reasons for seeking medical attention, and consumes considerable healthcare resources each year. The relation between etiology, underlying mechanisms and the specific symptoms and signs related to painful disorders is complex. Pain in an individual patient may be produced by more than one mechanism.

Chronic pain, such as pain present most of the time for a period of six months or longer during the prior year, is a highly pervasive complaint and consistently associated with psychological illness. Chronic pain may originate with a trauma, injury or infection, or there may be an ongoing cause of pain. Chronic pain may also present in the absence of any past injury or evidence of body damage. Common chronic pain can include headache, low back pain, cancer pain, arthritis pain, neurogenic pain (pain resulting from damage to the peripheral nerves or to the central nervous system), or psychogenic pain (pain not due to past disease or injury or any visible sign of damage inside or outside the nervous system).

Chronic pain may be treated or alleviated using medications, acupuncture, surgery, and neuromodulation therapy such as local electrical stimulation or brain stimulation, among others. Examples of neuromodulation include Spinal Cord Stimulation (SCS), Deep Brain Stimulation (DBS), Peripheral Nerve Stimulation (PNS), and Functional Electrical Stimulation (FES). Implantable neuromodulation systems have been applied to deliver such a therapy. An implantable neuromodulation system may include an implantable neurostimulator, also referred to as an implantable pulse generator (IPG), which can electrically stimulate tissue or nerve centers to treat nervous or muscular disorders. In an example, an IPG can deliver electrical pulses to a specific region in a patient's spinal cord, such as particular spinal nerve roots or nerve bundles, to create an analgesic effect that masks pain sensation.

### SUMMARY

By way of example, chronic pain management may involve determining appropriate treatment regimens such as SCS and evaluating therapy efficacy. Accurate pain assessment and characterization are desirable for managing patients with chronic pain. Currently, pain assessment generally relies on patient subjective report of pain symptoms, including severity, pattern, or duration of pain. Based on the patient reported pain sensation, a clinician may prescribe a pain therapy, such as to manually program an electrostimulator for delivering a neuromodulation therapy. However, the subjective description of pain sensation may be constrained by patient cognitive abilities. The subjective pain description may also be subject to intra-patient variation, such as due to a progression of a chronic disease, or a change in general health status or medication. Having a patient to report and describe each pain episode he or she has experienced is not efficient and may delay appropriate pain therapy. Additionally, for patients in an ambulatory setting who lack immediate access to medical assistance, manual adjustment of pain therapy by a clinician may not be feasible especially if immediate therapy titration is required. The present inventors have recognized that there remains a demand for improving pain management, such as systems and methods for objective pain assessment and automated closed-loop pain therapy based on objective pain assessment.

This document discusses, among other things, systems, devices, and methods for assessing pain in a subject. The system includes sensors to sense one or more physiological signals, and extract from the sensed physiological signals at least one cardiovascular parameter indicative of arterial pulsatile activity or cardiac electrical activity. The arterial pulsatile activity or cardiac electrical activity may be correlated to an increase in sympathetic tone when a pain episode occurs or when the chronic pain aggravates. The system may generate a pain score using the cardiovascular parameters. The pain score can be output to a patient or used for closed-loop control of a pain therapy.

Example 1 is a system for managing pain of a patient. The system comprises a sensor circuit, a pain analyzer circuit, and an output unit. The sensor circuit may be configured to sense at least one physiological signal. The pain analyzer circuit may be configured to measure, from each of the sensed at least one physiological signal, one or more cardiovascular parameters indicative of arterial pulsatile activity or cardiac electrical activity, and generate a pain score based on the measured one or more cardiovascular parameters. The output unit may be configured to output the pain score to a user or a process.

In Example 2, the subject matter of Example 1 optionally includes the sensor circuit that may be coupled to a first sensor configured to sense a first physiological event and a second sensor configured to sense a second physiological event. The second physiological event occurs temporally subsequent to the first physiological event. The one or more cardiovascular parameters may include a pulse wave transit parameter indicating arterial pulse wave propagation through a patient circulatory system during a period between the first and second physiologic events.

In Example 3, the subject matter of Example 2 optionally includes the pulse wave transit parameter that may include a pulse wave transit time (PWTT) elapsed from the first physiological event to the second physiological event. The pain analyzer circuit may be configured to generate the pain score based on a reduction of PWTT from a baseline PWTT.

In Example 4, the subject matter of Example 3 optionally includes the first sensor configured to sense an R wave in an electrocardiogram (ECG) signal, and the second sensor configured to sense an arterial pulse wave (APW) signal. The pain analyzer circuit may be configured to determine the PWTT based on an R-APW time interval between the sensed R wave and an APW onset indicating an onset of the arterial pulsatile activity.

In Example 5, the subject matter of Example 4 optionally may further comprise a third sensor configured to sense a heart sound (HS) signal. The pain analyzer circuit may be configured to determine a pre-ejection period (PEP) based on at least the sensed HS signal, and determine the PWTT based on a difference between the R-APW time interval and the PEP.

In Example 6, the subject matter of any one or more of Examples 2-5 optionally includes the first sensor configured to sense a heart sound (HS) signal, the second sensor configured to sense an arterial pulse wave (APW) signal, and the pain analyzer circuit configured to determine the PWTT based on a time interval between (1) a first (S1) HS component from the sensed HS signal and (2) an APW onset indicating an onset of the arterial pulsatile activity.

In Example 7, the subject matter of any one or more of Examples 2-6 optionally includes the first sensor configured to be positioned at or near a first location of an artery to sense the first physiological event indicative of arterial pulsatile activity at the first location, the second sensor configured to be positioned at or near a different second location of the artery to sense the second physiological event indicative of arterial pulsatile activity at the second location, and the pain analyzer circuit configured to determine the pulse wave transit parameter between the first and second physiological events.

In Example 8, the subject matter of any one or more of Examples 2-7 optionally includes the pulse wave transit parameter that may include a pulse wave velocity (PWV) indicative of a propagation speed of the arterial pulse wave between the first and second physiological events. The pain analyzer circuit may be configured to generate the pain score based on an increase of PWV from a baseline PWV.

In Example 9, the subject matter of any one or more of Examples 2-8 optionally includes at least one of the first or the second sensor that may include at least one of: a pressure sensor; a photoplethysmography (PPG) sensor; an impedance sensor; an accelerometer sensor; or a camera configured to capture an image indicative of arterial blood flow.

In Example 10, the subject matter of any one or more of Examples 1-9 optionally includes the pain analyzer circuit configured to measure the one or more cardiovascular parameters including a pulse wave morphological parameter.

In Example 11, the subject matter of any one or more of Examples 1-10 optionally includes the pain analyzer circuit configured to measure the one or more cardiovascular parameters including an electrocardiography (ECG) timing parameter or an ECG morphological parameter.

In Example 12, the subject matter of any one or more of Examples 1-11 optionally further comprises: an electrostimulator configured to generate electrostimulation energy to treat pain; and a controller circuit coupled to the pain analyzer circuit and the electrostimulator, the controller circuit configured to control the electrostimulator to deliver a pain therapy and to control the electrostimulation energy generated by the electrostimulator according to the pain score.

In Example 13, the subject matter of Example 12 optionally includes the electrostimulator that may further be configured to deliver at least one of: a spinal cord stimulation; a brain stimulation; or a peripheral nerve stimulation.

In Example 14, the subject matter of any one or more of Examples 12-13 optionally includes the controller circuit that may further be configured to deliver first electrostimulation to the patient in response to the pain score exceeding a threshold value, and to deliver second electrostimulation to the patient in response to the pain score falling below the threshold value. The first electrostimulation may differ from the second electrostimulation with respect to at least one of electrostimulation energy, an electrostimulation pulse shape, or an electrostimulation pattern.

In Example 15, the subject matter of any one or more of Examples 12-14 optionally includes an implantable neuromodulator device (IND) that includes one or more of the sensor circuit, the pain analyzer circuit, or the electrostimulator.

Example 16 is a method for managing pain of a patient using an implantable neuromodulator device (IND). The method comprises: sensing at least one physiological signal from the patient via a sensor circuit; measuring, from the sensed at least one physiological signal, one or more cardiovascular parameters indicative of arterial pulsatile activity or cardiac electrical activity; generating a pain score based on the measured one or more cardiovascular parameters; and outputting the pain score to a user or a process.

In Example 17, the subject matter of Example 16 optionally includes delivering a pain therapy via the IND. The pain therapy includes electrostimulation energy determined according to the pain score.

In Example 18, the subject matter of any one or more of Examples 16-17 optionally includes the sensed at least one physiological signal that may include a first physiological event and a second physiological event that occurs temporally subsequent to the first physiological event. The one or more cardiovascular parameters include a pulse wave transit parameter indicating an arterial pulse wave propagation through a patient circulatory system during a period between the first and second physiologic events.

In Example 19, the subject matter of Example 18 optionally includes the pulse wave transit parameter that may include a pulse wave transit time (PWTT) elapsed from the first physiological event to the second physiological event; and the pain score is generated based on a reduction of PWTT from a baseline PWTT.

In Example 20, the subject matter of Example 19 optionally includes: sensing a heart sound (HS) signals using a HS sensor; determining a pre-ejection period (PEP) based on at least the sensed HS signal; and measuring an R-APW time interval between an R wave of an electrocardiogram (ECG) and an arterial pulse wave (APW) onset indicating an onset of the arterial pulsatile activity. The PWTT may be determined as a difference between the R-APW time interval and the PEP.

In Example 21, the subject matter of any one or more of Examples 18-20 optionally includes the pulse wave transit parameter that may include a pulse wave velocity (PWV) indicative of a propagation speed of the arterial pulse wave between the first and second physiological events. The pain score may be generated based on an increase of PWV from a baseline PWV.

In Example 22, the subject matter of any one or more of Examples 16-21 optionally includes the one or more cardiovascular parameters that may include a pulse wave morphological parameter.

In Example 23, the subject matter of any one or more of Examples 16-22 optionally includes the one or more cardiovascular parameters that may include an electrocardiography (ECG) timing parameter or an ECG morphological parameter.

The pain score generated based on the cardiovascular parameters, such as the parameters indicative of cardia electrical activity or arterial pulsatile activity as discussed in this document, may improve medical diagnostics of automated characterization of patient pain, as well as individualized therapies to alleviate pain and to reduce side effects. The systems, devices, and methods discussed in this document may also enhance the performance and functionality of a pain management system or device. For example, through improved pain therapy based on patient individual need and therapy efficacy, battery longevity of an implantable device may be enhanced, or pain medication volume may be saved.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, a neuromodulation system and portions of an environment in which the neuromodulation system may operate.

FIG. 2 illustrates, by way of example and not limitation, a block diagram of a pain management system.

FIG. 3 illustrates, by way of example and not limitation, a block diagram of another pain management system.

FIG. 4 illustrates, by way of example and not limitation, a block diagram of a cardiovascular parameter generator configured to generate signal metrics for patient pain assessment.

FIG. 5 illustrates, by way of example and not limitation, a block diagram of a system for generating arterial pulse wave parameters.

FIG. 6 illustrates, by way of example and not limitation, a method for managing pain of a patient.

FIG. 7 illustrates, by way of example and not limitation, a method for quantizing pain using cardiovascular parameters.

FIG. 8 illustrates, by way of example and not limitation, a block diagram of an example machine upon which any one or more of the techniques discussed herein may perform.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

Clinically, patient pain may be associated with an increase in sympathetic tone. The increase in sympathetic activity may cause cardiovascular reactions, including constriction of peripheral blood vessels, increase in blood pressure, increase in heart rate, and increase in cardiac force of contraction, among others. Alterations in autonomic function such as increased sympathetic tone may also affect cardiac electrical activity, such as changes in electrocardiography (ECG) morphology or timing. Therefore, close monitoring of patient cardiovascular responses may provide an objective assessment of pain, and may be used to improve pain therapy efficacy.

Disclosed herein are systems, devices, and methods for or assessing pain in a subject, and optionally programming pain therapy based on the pain assessment. In various embodiments, the present system may include sensors configured to sense one or more physiological signals. A pain analyzer circuit may extract from the physiological signals cardiovascular parameters indicative of arterial pulsatile activity or cardiac electrical activity, and generate a pain score using at least the cardiovascular parameters. The system may include a neurostimulator that can adaptively control the delivery of pain therapy by automatically adjusting stimulation parameters based on the pain score.

The present system may be implemented using a combination of hardware and software designed to provide a closed-loop pain management regimen to increase therapeutic efficacy, increase patient satisfaction for neurostimulation therapies, reduce side effects, and/or increase device longevity. The present system may be applied in any neurostimulation (neuromodulation) therapies, including but not limited to SCS, DBS, PNS, FES, and Vagus Nerve Stimulation (VNS) therapies. In various examples, instead of providing closed-loop pain therapies, the systems, devices, and methods described herein may be used to monitor the patient and assess pain that either occurs intrinsically or is induced by nerve block procedures or radiofrequency ablation therapies, among others. The patient monitoring may include generating recommendations to the patient or a clinician regarding pain treatment.

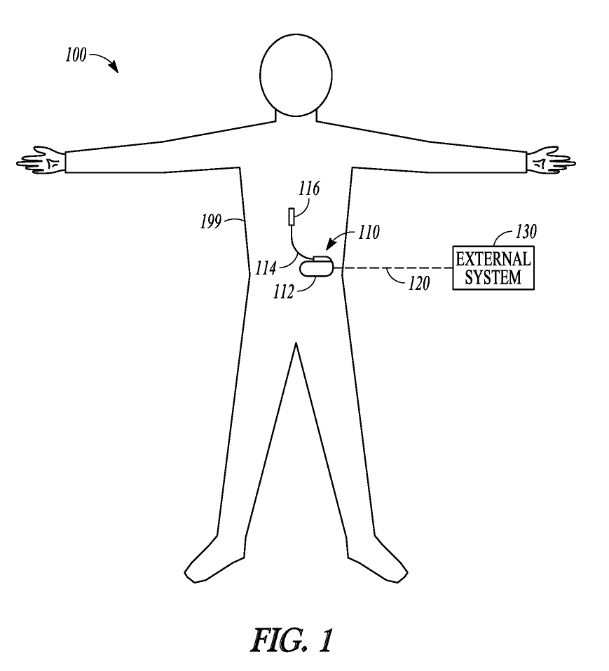

FIG. 1 illustrates, by way of example and not limitation, a neuromodulation system 100 for managing pain in a subject such as a patient with chronic pain, and portions of an environment in which the neuromodulation system 100 may operate. The neuromodulation system 100 may include an implantable system 110 that may be associated with a body 199 of the subject, and an external system 130 in communication with the implantable system 110 via a communication link 120.

The implantable system 110 may include an ambulatory medical device (AMD), such as an implantable neuromodulator device (IND) 112, a lead system 114, and one or more electrodes 116. The IND 112 may be configured for subcutaneous implant in a patient's chest, abdomen, or other parts of the body 199. The IND 112 may be configured as a monitoring and diagnostic device. The IND 112 may include a hermetically sealed can that houses sensing circuitry to sense physiological signals from the patient via sensing electrodes or ambulatory sensors associated with the patient and in communication with the IND 112. In some examples, the sensing electrodes or the ambulatory sensors may be included within the IND 112. One or more physiological or functional signals may be measured during a pain episode. The physiological or functional signals may be correlative to severity of the pain. The IND 112 may characterize and quantify the pain, such as to determine onset, intensity, severity, duration, or patterns of the pain experienced by the subject. The IND 112 may generate an alert to indicate occurrence of a pain episode, pain exacerbation, or efficacy of pain therapy, and present the alert to a clinician.

The IND 112 may alternatively be configured as a therapeutic device for treating or alleviating the pain. In addition to pain monitoring circuitry, the IND 112 may further include a therapy unit that can generate and deliver energy or modulation agents to a target tissue. The energy may include electrical, magnetic, or other types of energy. In some examples, the IND 112 may include a drug delivery system such as a drug infusion pump that can deliver pain medication to the patient, such as morphine sulfate or ziconotide, among others.

The IND 112 may include electrostimulation circuitry that generates electrostimulation pulses to stimulate a neural target via the electrodes 116 operably connected to the IND 112. In an example, the electrodes 116 may be positioned on or near a spinal cord, and the electrostimulation circuitry may be configured to deliver SCS to treat pain. In another example, the electrodes 116 may be surgically placed at other neural targets such as a brain or a peripheral neutral tissue, and the electrostimulation circuitry may be configured to deliver brain or peripheral stimulations. Examples of electrostimulation may include deep brain stimulation (DBS), trigeminal nerve stimulation, occipital nerve stimulation, vagus nerve stimulation (VNS), sacral nerve stimulation, sphenopalatine ganglion stimulation, sympathetic nerve modulation, adrenal gland modulation, baroreceptor stimulation, or transcranial magnetic stimulation, spinal cord stimulation (SCS), dorsal root ganglia (DRG) stimulation, motor cortex stimulation (MCS), transcranial direct current stimulation (tDCS), transcutaneous spinal direct current stimulation (tsDCS), pudendal nerve stimulation, multifidus muscle stimulation, transcutaneous electrical nerve stimulation (TENS), tibial nerve stimulation, among other peripheral nerve or organ stimulation. The IND 112 may additionally or alternatively provide therapies such as radiofrequency ablation (RFA), pulsed radiofrequency ablation, ultrasound therapy, high-intensity focused ultrasound (HIFU), optical stimulation, optogenetic therapy, magnetic stimulation, other peripheral tissue stimulation therapies, other peripheral tissue denervation therapies, or nerve blocks or injections.

In various examples, the electrodes 116 may be distributed in one or more leads of the lead system 114 electrically coupled to the IND 112. In an example, the lead system 114 may include a directional lead that includes at least some segmented electrodes circumferentially disposed about the directional lead. Two or more segmented electrodes may be distributed along a circumference of the lead. The actual number and shape of leads and electrodes may vary according to the intended application. Detailed description of construction and method of manufacturing percutaneous stimulation leads are disclosed in U.S. Pat. No. 8,019,439, entitled “Lead Assembly and Method of Making Same,” and U.S. Pat. No. 7,650,184, entitled “Cylindrical Multi-Contact Electrode Lead for Neural Stimulation and Method of Making Same,” the disclosures of which are incorporated herein by reference. The electrodes 116 may provide an electrically conductive contact providing for an electrical interface between the IND 112 and tissue of the patient. The neurostimulation pulses are each delivered from the IND 112 through a set of electrodes selected from the electrodes 116. In various examples, the neurostimulation pulses may include one or more individually defined pulses, and the set of electrodes may be individually definable by the user for each of the individually defined pulses.

Although the discussion herein with regard to the neuromodulation system 100 focuses on implantable device such as the IND 112, this is meant only by way of example and not limitation. It is within the contemplation of the present inventors and within the scope of this document, that the systems, devices, and methods discussed herein may also be used for pain management via subcutaneous medical devices, wearable medical devices (e.g., wrist watch, patches, garment- or shoe-mounted device), or other external medical devices, or a combination of implantable, wearable, or other external devices. The therapy, such as electrostimulation or medical therapies, may be used to treat various neurological disorders other than pain, which by way of example and not limitation may include epilepsy, obsessive compulsive disorder, tremor, Parkinson's disease, or dystonia, among other movement and affective disorders.

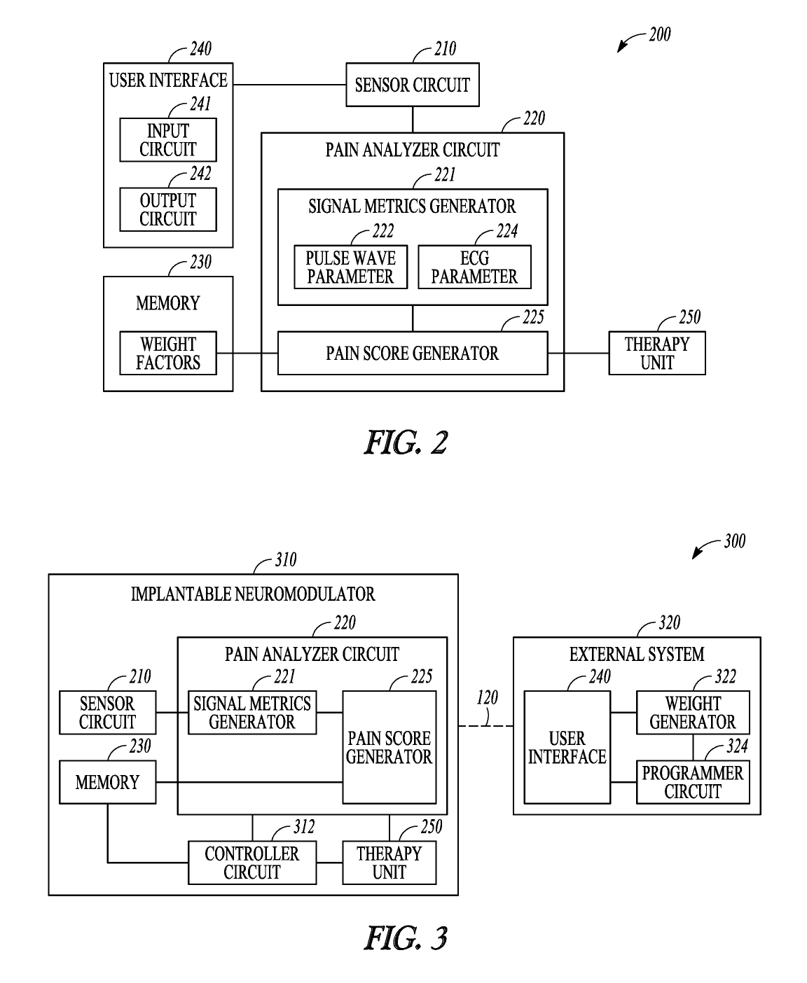

The external system 130 may be communicated with the IND 112 via a communication link 120. The external system 130 may include a dedicated hardware/software system such as a programmer, a remote server-based patient management system, or alternatively a system defined predominantly by software running on a standard personal computer. The external system 130 may be configured to control the operation of the IND 112, such as to program the IND 112 for delivering neuromodulation therapies. The external system 130 may additionally receive via the communication link 120 information acquired by IND 112, such as one or more physiological or functional signals. In an example, the external system 130 may determine a pain score based on the physiological or functional signals received from the IND 112, and program the IND 112 to deliver pain therapy in a closed-loop fashion. Examples of the external system and neurostimulation based on pain score are discussed below, such as with reference to FIGS. 2-3.

The communication link 120 may include one or more communication channels and intermediate devices between the external system and the IND, such as a wired link, a telecommunication link such as an internet connection, or a wireless link such as one or more of an inductive telemetry link, a radio-frequency telemetry link. The communication link 120 may provide for data transmission between the IND 112 and the external system 130. The transmitted data may include, for example, real-time physiological or functional signals acquired by and stored in the IND 112, therapy history data, data indicating device operational status of the IND 112, one or more programming instructions to the IND 112 which may include configurations for sensing physiologic signal or stimulation commands and stimulation parameters, or device self-diagnostic test, among others. In some examples, the IND 112 may be coupled to the external system 130 further via an intermediate control device, such as a handheld external remote control device to remotely instruct the IND 112 to generate electrical stimulation pulses in accordance with selected stimulation parameters produced by the external system 130.

Portions of the IND 112 or the external system 130 may be implemented using hardware, software, firmware, or combinations thereof. Portions of the IND 112 or the external system 130 may be implemented using an application-specific circuit that may be constructed or configured to perform one or more particular functions, or may be implemented using a general-purpose circuit that may be programmed or otherwise configured to perform one or more particular functions. Such a general-purpose circuit may include a microprocessor or a portion thereof, a microcontroller or a portion thereof, or a programmable logic circuit, or a portion thereof. For example, a “comparator” may include, among other things, an electronic circuit comparator that may be constructed to perform the specific function of a comparison between two signals or the comparator may be implemented as a portion of a general-purpose circuit that may be driven by a code instructing a portion of the general-purpose circuit to perform a comparison between the two signals.

FIG. 2 illustrates, by way of example and not limitation, a block diagram of a pain management system 200, which may be an embodiment of the neuromodulation system 100. The pain management system 200 may assess pain in a subject, and program pain therapy based on the pain assessment. As illustrated in FIG. 2, the pain management system 200 may include a sensor circuit 210, a pain analyzer circuit 220, a memory 230, and a user interface 240. The pain management system 200 may additionally include an optional therapy unit 250.

The sensor circuit 210 may be coupled to electrodes or various types of ambulatory sensors associated with the patient to sense at least one physiological signal from the patient. The sensor circuit 210 may include sense amplifier circuit that may pre-process the sensed signals, including, for example, amplification, digitization, filtering, or other signal conditioning operations. Various physiological signals, such as cardiac, pulmonary, neural, or biochemical signals may demonstrate characteristic signal properties in response to an onset, intensity, severity, duration, or patterns of pain. In an example, the sensor circuit 210 may be coupled to implantable or wearable sensors to sense cardiac signals such as electrocardiograph (ECG), intracardiac electrogram, gyrocardiography, magnetocardiography, heart rate signal, heart rate variability signal, cardiovascular pressure signal, or heart sounds signal, among others. In another example, the sensor circuit 210 may sense pulmonary signals such as a respiratory signal, a thoracic impedance signal, or a respiratory sounds signal. In still another example, the sensor circuit 210 may sense biochemical signals such as blood chemistry measurements or expression levels of one or more biomarkers, which may include, by way of example and not limitation, B-type natriuretic peptide (BNP) or N-terminal pro b-type natriuretic peptide (NT-proBNP), serum cytokine profiles, P2X4 receptor expression levels, gamma-aminobutyric acid (GABA) levels, TNFα and other inflammatory markers, cortisol, adenosine, Glial cell-derived neurotrophic factor (GDNF), Nav 1.3, Nav 1.7, or Tetrahydrobiopterin (BH4) levels, among other biomarkers.

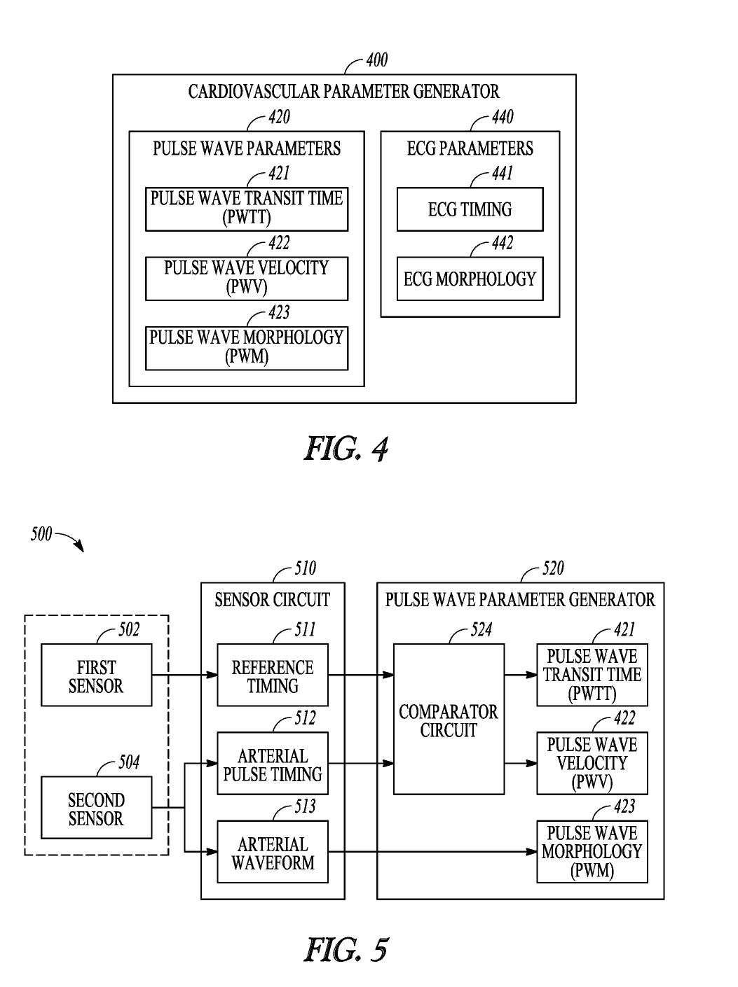

In an example, the sensed physiological signal may contain information corresponding to arterial pulsatile activity or cardiac electrical activity. Pain, through automatic nervous system, may cause cardiovascular reactions such as increased heart rate, enhanced cardiac force, and changes in electrical activity such as changes in electrocardiography (ECG) morphology or timing. Information corresponding to arterial pulsatile activity or cardiac electrical activity may be used to characterize and quantify patient pain. Examples of the cardiovascular signals in pain quantification are discussed below, such as with reference to FIG. 4.

The pain analyzer circuit 220 may generate a pain score based on at least the sensed at least one physiological signal indicative of patient arterial pulsatile activity or cardiac electrical activity. The pain analyzer circuit 220 may be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general purpose processor that may receive and execute a set of instructions of performing the functions, methods, or techniques described herein.

The pain analyzer circuit 220 may include circuit sets comprising one or more other circuits or sub-circuits that may, alone or in combination, perform the functions, methods or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

As illustrated in FIG. 2, the pain analyzer circuit 220 may include a signal metrics generator 221 and a pain score generator 225. The signal metrics generator 221 may generate signal metrics from the sensed at least one physiological signal. The signal metrics may include statistical parameters extracted from the sensed signal, such as signal mean, median, or other central tendency measures or a histogram of the signal intensity, among others. The signal metrics may additionally or alternatively include morphological parameters such as maximum or minimum within a specified time period such as a cardiac cycle, positive or negative slope or higher order statistics, or signal power spectral density at a specified frequency range, among other morphological parameters. The signal metrics may additionally include timing information such as a time interval between a first characteristic point in one signal and a second characteristic point in another signal. In various examples, the signal metrics generator 221 may generate from the sensed at least one physiological signal a plurality of cardiovascular parameters, including one or more of a pulse wave parameter 222 or an electrocardiography (ECG) parameter 224. The pulse wave parameter 222 may be indicative of patient arterial pulsatile activity. Examples of the pulse wave parameter 222 may include a pulse wave transit parameter, such as a pulse wave transit time (PWTT) or a pulse wave velocity (PWV), which represent characteristics of conduction of the arterial pulse through a patient circulatory system, or a pulse wave morphology (PWM) parameter which represents pressure distribution or blood volume dynamics when the blood propagates along the vascular system. The ECG parameter 224 may be indicative of patient cardiac electrical activity. The cardiovascular parameters such as the pulse wave parameter 222 or the ECG parameter 224 may be correlated to the patient pain such as through a sympathetic and parasympathetic nervous system control, and can therefore be used to quantify the patient pain. Examples of the cardiovascular parameters used for pain quantification are discussed below, such as with reference to FIG. 4.

The pain score generator 225 may generate a pain score using the measurements of the signal metrics. The pain score can be represented as a numerical or categorical value that quantifies the patient's overall pain symptom. In an example, a composite signal metric may be generated using a linear or nonlinear combination of a plurality of the signal metrics respectively weighted by weight factors. The pain score generator 225 may compare the composite signal metric to one or more threshold values or range values, and assign a corresponding pain score (such as numerical values from 0 to 10) based on the comparison.

In another example, the pain score generator 225 may compare each of the signal metrics to respectively specified threshold or range values, assign a corresponding signal metric-specific pain score based on the comparison to the threshold, and compute a composite based pain score using a linear or nonlinear fusion of the signal metric-specific pain scores weighted by their respective weight factors. In an example, the threshold can be inversely proportional to signal metric's sensitivity to pain. A signal metric that is more sensitive to pain may have a corresponding lower threshold and a larger metric-specific pain score, thus plays a more dominant role in the composite based pain score than another signal metric that is less sensitive to pain. Examples of the fusion algorithm may include weighted averages, voting, decision trees, or neural networks, among others. The pain score generated by the pain score generator 225 may be output to a system user or a process.

In some example, the pain score generator 225 may generate a metric-specific pain score based on a comparison of a cardiovascular parameter Pi, such as an arterial pulse waveform parameter or the ECG waveform parameter, to a template Ti. The template Ti may be an individualized or population-based representative cardiovascular parameter when the patient experiences pain. The template Ti thus formed can be referred to as a representative “pain template”. Alternatively, the template Ti may be a “pain-free template” formed using individualized or population-based arterial pulse waveform parameter or the ECG waveform parameter when the patient experiences no known pain. In an example, the cardiovascular parameter may include morphology of at least a portion of the atrial pulse waveform or morphology of at least a portion of ECG waveform. The arterial pulse or ECG morphological template Ti may be formed from an individual patient baseline, or from a population database. The pain score generator 225 may compute a similarity measure S(Pi,Ti) between the Pi and the respective morphological template Ti, and determine the metric-specific pain score based on the similarity measure. Examples of the similarity measure may include distance in a normed vector space (such as L1 norm, L2 norm or Euclidian distance, and infinite norm), correlation coefficient, mutual information, or ratio image uniformity, among others.

The metric-specific pain score Xi may be determined as a function of the similarity measure S(Pi,Ti), that is, Xi=f (S(Pi, Ti)), where f is linear or nonlinear function. In an example, the X_{Fi} may be proportional to the similarity measure S(Pi,Ti), that is, Xi=k*(S(Pi, Ti), where k is a positive coefficient. A higher pain score may be assigned for the cardiovascular parameter Fi if it highly resembles the individualized or universal template of the pain image. In a similar fashion, the pain score generator 225 may generate additional metric-specific pain score Xj pertaining to a different cardiovascular parameter Fj. The pain score generator 225 may generate a composite pain score X such as a weighted combination of Xi and Xj.

In various examples, in addition to the cardiovascular parameters, the sensor circuit 210 may sense functional signals from the patient. Examples of the functional signals may include, but not limited to, patient posture, gait, balance, or physical activity signals, among others. The sensor circuit 210 may sense the functional signals via one or more implantable or wearable motion sensors, including an accelerometer, a gyroscope (which may be a one-, two-, or three-axis gyroscope), a magnetometer (e.g., a compass), an inclinometer, a goniometer, a electromagnetic tracking system (ETS), or a global positioning system (GPS) sensor, among others. Detailed description of functional signals for use in pain characterization are disclosed in commonly assigned U.S. Provisional Patent Application Ser. No. 62/445,075, entitled “PAIN MANAGEMENT BASED ON FUNCTIONAL MEASUREMENTS”, the disclosures of which are incorporated herein by reference. Commonly assigned U.S. Provisional Patent Application Ser. No. 62/445,061, entitled “PAIN MANAGEMENT BASED ON BRAIN ACTIVITY MONITORING” describes information of brain activity for use in pain analysis, the disclosure of which is incorporated herein by reference in its entirety. Commonly assigned U.S. Provisional Patent Application Ser. No. 62/445,069, entitled “PAIN MANAGEMENT BASED ON RESPIRATION-MEDIATED HEART RATES” describes information of respiration-mediated heart rate for use in pain analysis, the disclosure of which is incorporated herein by reference in its entirety. Commonly assigned U.S. Provisional Patent Application Ser. No. 62/445,082, entitled “PAIN MANAGEMENT BASED ON EMOTIONAL EXPRESSION MEASUREMENTS” describes measurements of patient emotional expressions for use in pain analysis, the disclosure of which is incorporated herein by reference in its entirety. Commonly assigned U.S. Provisional Patent Application Ser. No. 62/445,092, entitled “PAIN MANAGEMENT BASED ON MUSCLE TENSION MEASUREMENTS” describes measurements of patient muscle tension including electromyography for use in pain analysis, the disclosure of which is incorporated herein by reference in its entirety. One or more of these additional signals or measurements may be used by the pain analyzer circuit 220 to generate a pain score.

The signal metrics generator 221 may generate functional signal metrics from functional signals, and the pain score generator 225 may determine the pain score using a linear or nonlinear combination of the cardiovascular parameters and the functional signal metrics.

The memory 230 may be configured to store sensor signals or signal metrics such as generated by the sensor circuit 210 and the signal metrics generator 221, and the pain scores such as generated by the pain score generator 225. Data storage at the memory 230 may be continuous, periodic, or triggered by a user command or a specified event. In an example, as illustrated in FIG. 2, the memory 230 may store weight factors, which may be used by the pain score generator 225 to generate the pain score. The weight factors may be provided by a system user, or alternatively be automatically determined or adjusted such as based on the corresponding signal metrics' reliability in representing an intensity of the pain. Examples of the automatic weight factor generation are discussed below, such as with reference to FIG. 3.

The user interface 240 may include an input circuit 241 and an output unit 242. In an example, at least a portion of the user interface 240 may be implemented in the external system 130. The input circuit 241 may enable a system user to program the parameters used for sensing the physiological signals, generating signal metrics, or generating the pain score. The input circuit 241 may be coupled on one or more input devices such as a keyboard, on-screen keyboard, mouse, trackball, touchpad, touch-screen, or other pointing or navigating devices. In some example, the input circuit may be incorporated in a mobile device such as a smart phone or other portable electronic device with a mobile application (“App”). The mobile App may enable a patient to provide pain description or quantified pain scales during the pain episodes. In an example, the input circuit 241 may enable a user to confirm, reject, or edit the programming of the therapy unit 250, such as parameters for electrostimulation, as to be discussed in the following.

The output unit 242 may include a display to present to a system user such as a clinician the pain score. The output unit 242 may also display information including the physiological or functional signals, trends of the signal metric, or any intermediary results for pain score calculation such as the signal metric-specific pain scores. The information may be presented in a table, a chart, a diagram, or any other types of textual, tabular, or graphical presentation formats, for displaying to a system user. The presentation of the output information may include audio or other human-perceptible media format. In an example, the output unit 242 may generate alerts, alarms, emergency calls, or other forms of warnings to signal the system user about the pain score. In some examples, the alert may indicate an elevated blood pressure in response to the pulse wave transit parameter satisfying a specified condition, such as the PWTT falling below a specified threshold, or the PWV exceeding a specified threshold.

The optional therapy circuit 250 may be configured to deliver a therapy to the patient in response to the pain score. In an example, the therapy circuit 250 may include an electrostimulator configured to generate electrostimulation energy to treat pain. In an example, the electrostimulator may deliver spinal cord stimulation (SCS) via electrodes electrically coupled to the electrostimulator. The electrodes may be surgically placed at a region at or near a spinal cord tissue, which may include, by way of example and not limitation, dorsal column, dorsal horn, spinal nerve roots such as the dorsal nerve root, and dorsal root ganglia. The SCS may be in a form of stimulation pulses that are characterized by pulse amplitude, pulse width, stimulation frequency, duration, on-off cycle, pulse shape or waveform, temporal pattern of the stimulation, among other stimulation parameters. Examples of the stimulation pattern may include burst stimulation with substantially identical inter-pulse intervals, or ramp stimulation with incremental inter-pulse intervals or with decremental inter-pulse intervals. In some examples, the frequency or the pulse width may change from pulse to pulse. The electrostimulator may additionally or alternatively deliver electrostimulation to other target tissues such as peripheral nerves tissues. In addition to or in lieu of the SCS, other electrostimulation type of pain therapy may be delivered, which may include deep brain stimulation (DBS), functional electrical stimulation (FES), vagus nerve stimulation (VNS), or peripheral nerve stimulation (PNS) at various locations including trigeminal nerve stimulation, occipital nerve stimulation, sacral nerve stimulation, sphenopalatine ganglion stimulation, sympathetic modulation, adrenal gland modulation, baroreceptor stimulation, or transcranial magnetic stimulation. In an example, the electrostimulator may deliver transcutaneous electrical nerve stimulation (TENS) via detachable electrodes that are affixed to the skin.

The therapy circuit 250 may additionally or alternatively include a drug delivery system, such as an intrathecal drug delivery pump that may be surgically placed under the skin, which may be programmed to inject medication or biologics through a catheter to the area around the spinal cord. Other examples of drug delivery system may include a computerized patient-controlled analgesia pump that may deliver the prescribed pain medication to the patient such as via an intravenous line. In some examples, the therapy circuit 250 may be delivered according to the pain score received from the pain score generator 225.

FIG. 3 illustrates, by way of example and not limitation, another example of a pain management system 300, which may be an embodiment of the neuromodulation system 100 or the pain management system 200. The pain management system 300 may include an implantable neuromodulator 310 and an external system 320, which may be, respectively, embodiments of the IND 112 and the external system 130 as illustrated in FIG. 1. The external system 320 may be communicatively coupled to the implantable neuromodulator 310 via the communication link 120.

The implantable neuromodulator 310 may include several components of the pain management system 200 as illustrated in FIG. 2, including the sensor circuit 210, the pain analyzer circuit 220, the memory 230, and the therapy unit 250. As discussed with reference to FIG. 2, the pain analyzer circuit 220 includes the pain score generator 225 that determines a pain score using weight factors stored in the memory 230 and the signal metrics from the signal metrics generator 221 which may also be included in the pain analyzer circuit 220. The implantable neuromodulator 310 may include a controller circuit 312, coupled to the therapy unit 250, which controls the generation and delivery of pain therapy, such as neurostimulation energy. The controller circuit 312 may control the generation of electrostimulation pulses according to specified stimulation parameters. The stimulation parameters may be provided by a system user. Alternatively, the stimulation parameters may be automatically determined based on the intensity, severity, duration, or pattern of pain, which may be subjectively described by the patient or automatically quantified based on the physiological or functional signals sensed by the sensor circuit 210. For example, when a patient-described or sensor-indicated quantification exceeds a respective threshold value or falls within a specified range indicating elevated pain, the electrostimulation energy may be increased to provide stronger pain relief. Increased electrostimulation energy may be achieved by programming a higher pulse intensity, a higher frequency, or a longer stimulation duration or “on” cycle, among others. Conversely, when a patient-described or sensor-indicated pain quantification falls below a respective threshold value or falls within a specified range indicating no pain or mild pain, the electrostimulation energy may be decreased. The controller circuit 312 may also adjust stimulation parameters to alleviate side effects introduced by the electrostimulation of the target tissue.

Additionally or alternatively, the controller circuit 312 may control the therapy unit 250 to deliver electrostimulation pulses via specified electrodes. In an example of pain management via SCS, a plurality of segmented electrodes, such as the electrodes 116, may be distributed in one or more leads. The controller circuit 312 may configure the therapy unit 250 to deliver electrostimulation pulses via a set of electrodes selected from the plurality of electrodes. The electrodes may be manually selected by a system user, or automatically selected based on the pain score.

The implantable neuromodulator 310 may receive the information about electrostimulation parameters and the electrode configuration from the external system 320 via the communication link 120. Additional parameters associated with operation of the therapy unit 250, such as battery status, lead impedance and integrity, or device diagnostic of the implantable neuromodulator 310, may be transmitted to the external system 320. The controller circuit 312 may control the generation and delivery of electrostimulation using the information about electrostimulation parameters and the electrode configuration from the external system 320. Examples of the electrostimulation parameters and electrode configuration may include: temporal modulation parameters such as pulse amplitude, pulse width, pulse rate, or burst intensity; morphological modulation parameters respectively defining one or more portions of stimulation waveform morphology such as amplitude of different phases or pulses included in a stimulation burst; or spatial modulation parameters such as selection of active electrodes, electrode combinations which define the electrodes that are activated as anodes (positive), cathodes (negative), and turned off (zero), and stimulation energy fractionalization which defines amount of current, voltage, or energy assigned to each active electrode and thereby determines spatial distribution of the modulation field.

In an example, the controller circuit 312 may control the generation and delivery of electrostimulation in a closed-loop fashion by adaptively adjusting one or more stimulation parameters or stimulation electrode configuration based on the pain score. For example, if the pain score exceeds the pain threshold (or falls within a specified range indicating an elevated pain), then the first electrostimulation may be delivered. Conversely, if the composite pain score falls below a respective threshold value (or falls within a specified range indicating no pain or mild pain), then a second pain therapy, such as second electrostimulation may be delivered. The first electrostimulation may differ from the second electrostimulation with respect to at least one of the stimulation energy, pulse amplitude, pulse width, stimulation frequency, duration, on-off cycle, pulse shape or waveform, electrostimulation pattern such as electrode configuration or energy fractionalization among active electrodes, among other stimulation parameters. In an example, the first electrostimulation may have higher energy than the second electrostimulation, such as to provide stronger effect of pain relief. Examples of increased electrostimulation energy may include higher pulse intensity, a higher frequency, or a longer stimulation duration or “on” cycle, among others.

The parameter adjustment or stimulation electrode configuration may be executed continuously, periodically at specified time, duration, or frequency, or in a commanded mode upon receiving from a system user a command or confirmation of parameter adjustment. In some examples, the closed-loop control of the electrostimulation may be further based on the type of the pain, such as chronic or acute pain. In an example, the pain analyzer circuit 220 may trend the signal metric over time to compute an indication of abruptness of change of the signal metrics, such as a rate of change over a specified time period. The pain episode may be characterized as acute pain if the signal metric changes abruptly (e.g., the rate of change of the signal metric exceeding a threshold), or as chronic pain if the signal metric changes gradually (e.g., the rate of change of the signal metric falling below a threshold). The controller circuit 312 may control the therapy unit 250 to deliver, withhold, or otherwise modify the pain therapy in accordance with the pain type. For example, incidents such as toe stubbing or bodily injuries may cause abrupt changes in certain signal metrics, but no adjustment of the closed-loop pain therapy is deemed necessary. On the contrary, if the pain analyzer circuit 220 detects chronic pain characterized by gradual signal metric change, then the closed-loop pain therapy may be delivered accordingly.

The external system 320 may include the user interface 240, a weight generator 322, and a programmer circuit 324. The weight generator 322 may generate weight factors used by the pain score generator 225 to generate the pain score. The weight factors may indicate the signal metrics' reliability in representing an intensity of the pain. A sensor metric that is more reliable, or more sensitive or specific to the pain, would be assigned a larger weight than another sensor metric that is less reliable, or less sensitive or specific to the pain. In an example, the weight factors may be proportional to correlations between a plurality of quantified pain scales (such as reported by a patient) and measurements of the measurements of the signal metrics corresponding to the plurality of quantified pain scales. A signal metric that correlates with the pain scales is deemed a more reliable signal metric for pain quantification, and is assigned a larger weight factor than another signal metric less correlated with the quantified pain scales. In another example, the weight generator 322 may determine weight factors using the signal sensitivity to pain. The signal metrics may be trended over time, such as over approximately six months. The signal sensitivity to pain may be represented by a rate of change of the signal metrics over time during a pain episode. The signal sensitivity to pain may be evaluated under a controlled condition such as when the patient posture or activity is at a specified level or during specified time of the day. The weight generator 322 may determine weight factors to be proportional to signal metric's sensitivity to pain.

The programmer circuit 324 may produce parameter values for operating the implantable neuromodulator 310, including parameters for sensing physiological and functional signals and generating signal metrics, and parameters or electrode configurations for electrostimulation. In an example, the programmer circuit 324 may generate the stimulation parameters or electrode configurations for SCS based on the pain score produced by the pain score generator 225. Through the communication link 120, the programmer circuit 324 may continuously or periodically provide adjusted stimulation parameters or electrode configuration to the implantable neuromodulator 210A. By way of non-limiting example and as illustrated in FIG. 3, the programmer circuit 324 may be coupled to the user interface 234 to allow a user to confirm, reject, or edit the stimulation parameters, sensing parameters, or other parameters controlling the operation of the implantable neuromodulator 210A. The programmer circuit 324 may also adjust the stimulation parameter or electrode configuration in a commanded mode upon receiving from a system user a command or confirmation of parameter adjustment.

The programmer circuit 324, which may be coupled to the weight generator 322, may initiate a transmission of the weight factors generated by the weight generator 322 to the implantable neuromodulator 310, and store the weight factors in the memory 230. In an example, the weight factors received from the external system 320 may be compared to previously stored weight factors in the memory 230. The controller circuit 312 may update the weight factors stored in the memory 230 if the received weight factors are different than the stored weights. The pain analyzer circuit 220 may use the updated weight factors to generate a pain score. In an example, the update of the stored weight factors may be performed continuously, periodically, or in a commanded mode upon receiving a command from a user. In various examples, weight factors may be updated using a fusion model. Commonly assigned U.S. Provisional Patent Application Ser. No. 62/445,095, entitled “PATIENT-SPECIFIC CALIBRATION OF PAIN QUANTIFICATION” describes systems and methods for calibrating a fusion model, such as adjusting weights for signal metrics, using a reference pain quantification, the disclosure of which is incorporated herein by reference in its entirety.

In some examples, the pain score may be used by a therapy unit (such as an electrostimulator) separated from the pain management system 300. In various examples, the pain management system 300 may be configured as a monitoring system for pain characterization and quantification without delivering closed-loop electrostimulation or other modalities of pain therapy. The pain characterization and quantification may be provided to a system user such as the patient or a clinician, or to a process including, for example, an instance of a computer program executable in a microprocessor. In an example, the process includes computer-implemented generation of recommendations or an alert to the system user regarding pain medication (e.g., medication dosage and time for taking a dose), electrostimulation therapy, or other pain management regimens. The therapy recommendations or alert may be based on the pain score, and may be presented to the patient or the clinician in various settings including in-office assessments (e.g. spinal cord stimulation programming optimization), in-hospital monitoring (e.g. opioid dosing during surgery), or ambulatory monitoring (e.g. pharmaceutical dosing recommendations).

In an example, in response to the pain score exceeding a threshold which indicates elevated pain symptom, an alert may be generated and presented at the user interface 240 to remind the patient to take pain medication. In another example, therapy recommendations or alerts may be based on information about wearing-off effect of pain medication, which may be stored in the memory 230 or received from the user interface 240. When the drug effect has worn off, an alert may be generated to remind the patient to take another dose or to request a clinician review of the pain prescription. In yet another example, before a pain therapy such as neurostimulation therapy is adjusted (such as based on the pain score) and delivered to the patient, an alert may be generated to forewarn the patient or the clinician of any impending adverse events. This may be useful as some pain medication may have fatal or debilitating side effects. In some examples, the pain management system 300 may identify effect of pain medication addiction such as based on functional and physiological signals. An alert may be generated to warn the patient about effects of medication addiction and thus allow medical intervention.

FIG. 4 illustrates, by way of example and not limitation, a block diagram of a cardiovascular parameter generator 400 configured to generate signal metrics for patient pain assessment. The cardiovascular parameter generator 400 is an embodiment of the signal metrics generator 221 as illustrated in FIG. 2. By way of example and not limitation, the parameters generated by the cardiovascular parameter generator 400 may include one or more pulse wave parameters 420, and one or more ECG parameters 440, which may be embodiments of the pulse wave parameters 222 and ECG parameters 224, respectively.

The pulse wave parameters 420 may include timing parameters, statistical parameters, or morphological parameters obtained from an arterial pulse waveform. By way of example and not limitation, the pulse wave parameters 420 may include one or more of a pulse wave transit time (PWTT) parameter 421, a pulse wave velocity (PWV) parameter 422, or a pulse wave morphology (PWM) parameter 423. The PWTT parameter 421 may represent time it takes an arterial pressure waveform to propagate from one location to another location of the vascular system, such as through a length of the arterial tree. The PWTT may be measured using two sensors respectively measuring time or arrival of the pulse wave at two different locations. The PWV parameter 422 may represent a propagation speed of the arterial pulse wave along a length of the arterial tree, such as between two physiological events respectively detected at two different locations of the vascular system. The PWM parameter 423 may include morphological metrics extracted from the arterial pulse waveform. An arterial pulse waveform, corresponding to one heart contraction that includes a systole and diastole, may include consecutive temporal phases of systolic upstroke, systolic peak, systolic decline, dicrotic notch, dicrotic runoff, and end-diastolic pressure. The PWM parameter 423 may include signal intensity (amplitude or power), change or rate of change of signal intensity at the different temporal phases of the arterial pulse waveform. Examples of the PWM parameters 423 may include systolic pressure intensity, diastolic pressure intensity, area under the arterial pulse waveform, direct-current component of arterial pulse waveform, filtered arterial pulse waveform such as at specific frequency bands, dicrotic notch amplitude, or time interval between systolic and diastolic peaks, among others. The PWM parameters may be correlated to several cardiovascular features innervated by sympathetic nervous system. For example, the rate of change (i.e., the slope) of the systolic upstroke phase of an arterial pulse waveform may be correlated to myocardial contractility and systemic vascular resistance. A steeper systolic upstroke may indicate an enhanced myocardial contractility, an elevated sympathetic tone, and may be associated with an occurrence of a pain episode or aggravated pain. Examples of the arterial pulse waveform measurement and parameter extraction are discussed below, such as with reference to FIG. 5.

The ECG parameters 440 may include timing parameters, statistical parameters, or morphological parameters obtained from an ECG signal. By way of example and not limitation, the ECG parameters 440 may include one or more of an ECG timing parameter 441 and an ECG morphology parameter 442. Examples of the ECG timing parameter 441 may include P wave to P wave (P-P) interval representing duration between the onset of two consecutive atrial depolarizations, P wave to R wave (P-R) interval representing a duration between the onset of atrial depolarization to the onset of ventricular depolarization, QRS duration representing a duration of ventricular depolarization, Q wave to T wave (Q-T) interval representing a duration from the beginning of the QRS complex (ventricular depolarization) to the end of the T wave (ventricular repolarization), or ST segment duration representing a duration from the end of ventricular depolarization to the onset of ventricular repolarization, among others. The ECG morphology parameter 442 may include amplitude of QRS, slope of R wave, or ST segment elevation. The ECG morphological parameter 442 may additionally or alternatively include parameters extracted from vectorcardiogram (VCG), which is a three-dimensional representation of multi-lead ECG. An example of the ECG morphology parameter 442 based on the VCG may include a spatial QRS-T angle (SA), which represents an angle of deviation between the QRS-axis representing ventricular depolarization and the T-axis representing ventricular repolarization. The SA is indicative of the difference in orientation between the ventricular depolarization and repolarization. In some examples, the multi-lead ECG in Cartesian coordinates can be transformed to a two-dimensional polar coordinate system, or a three-dimensional spherical coordinate system, and the ECG morphological parameters 442 may be extracted from the representations in the polar or spherical coordinate system. Each point of the ECG presentation in the spherical coordinate system may be represented by magnitude, elevation angle, and azimuth angle. Examples of the ECG morphological parameters 442 may include QRS complex azimuth angle, QRS complex elevation angle, T wave azimuth angle, or T wave elevation angle, among others.

In some examples, the pulse wave parameters 420 or the ECG parameters 440 may include frequency-domain features such as power spectra at specified frequency bands, spectral entropy, frequency modulation of speech, or other transformed-domain features such as obtained from wavelet decomposition or signal filtering through a filter bank. In some examples, the feature extraction and recognition may include reducing the feature dimensionality through feature space projection such as a principal component analysis (PCA). The pulse wave parameters 420 or the ECG parameters 440 may be provide to the pain score generator 225 to generate a pain score.

FIG. 5 illustrates, by way of example and not limitation, a system 500 for generating arterial pulse wave parameters. The system 500 may be an embodiment of the pain management system 200 as illustrated in FIG. 2. The system 500 may include a sensor circuit 510 which is an embodiment of the sensor circuit 210, and a pulse wave parameter generator 520 which is an embodiment of the cardiovascular parameter generator 400.

The sensor circuit 510 may be communicatively coupled to a first sensor 502 and a second sensor 504. The first sensor 502 may be configured to sense a first physiological event, and the second sensor 504 may be configured to sense a different second physiological event that occurs temporally subsequent to the first physiological event. The sensor circuit 510 may include sense amplifier circuits that may pre-process the sensed physiological signals. The sensor circuit 510 may generate a reference timing 511 from the pre-processed first physiological signal, and generate an arterial pulse timing 512 and arterial pulse waveform 513 from the pre-processed second physiological signal.

The second sensor 504 may include an ambulatory sensor configured to non-invasively measure the arterial pulsatile activity from a specific artery, such as a common iliac artery, an internal iliac artery, a gonadal artery, an inferior mesenteric artery, an inferior rectal artery, an inferior gluteal artery, a superior gluteal artery, a renal artery, or a femoral artery, among others. Examples of the second sensor 504 for sensing arterial pulsatile activity may include a pressure sensor, a photoplethysmography (PPG) sensor, an impedance sensor, or an accelerometer sensor, among other sensors.

The pulse wave parameter generator 520 may be configured to generate one or more pulse wave transit parameters during a period between the first and second physiologic events. As illustrated in FIG. 5, the pulse wave parameter generator 520 may include a comparator circuit 524 that may compare the reference timing 511 and the arterial pulse timing 512. Based on that comparison, the pulse wave parameter generator 520 may measure the pulse wave transit time (PWTT) 421 elapsed from the first physiological event to the second physiological event, or the pulse wave velocity (PWV) 422 representing a propagation speed of the arterial pulse wave between the first and second physiological events.

In an example, the first sensor is configured to sense an R wave in an ECG signal, and the second sensor is configured to sense an arterial pulse wave (APW) signal. The pulse wave parameter generator 520 may determine pulse wave transmit parameter (such as PWTT 421 or PWV 422) based on a time interval (R-APW interval) between the sensed R wave and an APW onset indicating an onset of the arterial pulsatile activity. In another example, the pulse wave parameter generator 520 may determine the pulse wave transit parameter based on a difference between the R-APW interval and a pre-ejection period (PEP). The PEP represents the time period between when the ventricular contraction occurs and the semilunar valves open and blood ejection into the aorta commences. By subtracting the PEP from the R-APW interval, the resulting time period may be more specific to the conduction of arterial pulse wave through the artery originated from the aorta. In an example, a third sensor may be configured to sense a heart sound (HS) signal. Examples of the HS sensor may include lead-based, device-associated, or standalone accelerometers or microphone sensors for sensing HS. The sensor circuit 510 may sense from the HS signal a first heart sound (S1) timing, and the pulse wave parameter generator 520 may determine the PEP as a time interval between the R wave and S1 heart sound from the sensed HS signal within the same cardiac cycle.

In an example, the first sensor is configured to sense a heart sound (HS) signal, and the second sensor is configured to sense an arterial pulse wave (APW) signal. The pulse wave parameter generator 520 may determine pulse wave transmit parameter (such as PWTT or PWV) based on a time interval) a first (S1) HS component from the sensed HS signal and an APW onset indicating an onset of the arterial pulsatile activity. In yet another example, the first sensor is configured to be positioned at or near a first location of an artery to sense the first physiological event indicative of arterial pulsatile activity at the first location, and the second sensor is configured to be positioned at or near a different second location of the artery to sense the second physiological event indicative of arterial pulsatile activity at the second location. In an example, the first and second physiological events are respective wavefronts (APW1 and APW2) of the arterial pulse wave detected at the first and second locations. The pulse wave parameter generator 520 may determine the pulse wave transmit parameter (such as PWTT 421 or PWV 422) based on the time elapsed between the APW1 and APW2.

In some examples, the first sensor 502 or the second sensor 504 may include a camera configured to capture an image indicative of arterial blood flow. The camera may be an implantable, wearable, or otherwise ambulatory camera associated with the patient. In an example, the camera may capture an image or a video sequence of arterial blood flow from two separated locations along the length of an arterial tree. In another example, two separate cameras may be respectively disposed at two locations along the length of the arterial tree to simultaneously capture an image or a video sequence of the arterial blood flow at the two locations. The captured images or video sequences may be processed, including spatial decomposition and temporal filtering. The processed images may be amplified to reveal periodic color variation (e.g., change in redness of a skin as blood flows through the artery), which is indicative of arterial pulsatile activity. The pulse wave parameter generator 520 may determine the pulse wave transmit parameter (such as PWTT 421 or PWV 422) based on the color variation of the image of arterial blood flow.

The pulse wave parameter generator 520 may additionally or alternatively generate the pulse wave morphology (PWM) parameter based on the arterial pulse waveform 513. One or more of the PWTT 421, the PWV 422, or the PWM 423 may be used by the pain score generator 225 to generate a pain score.

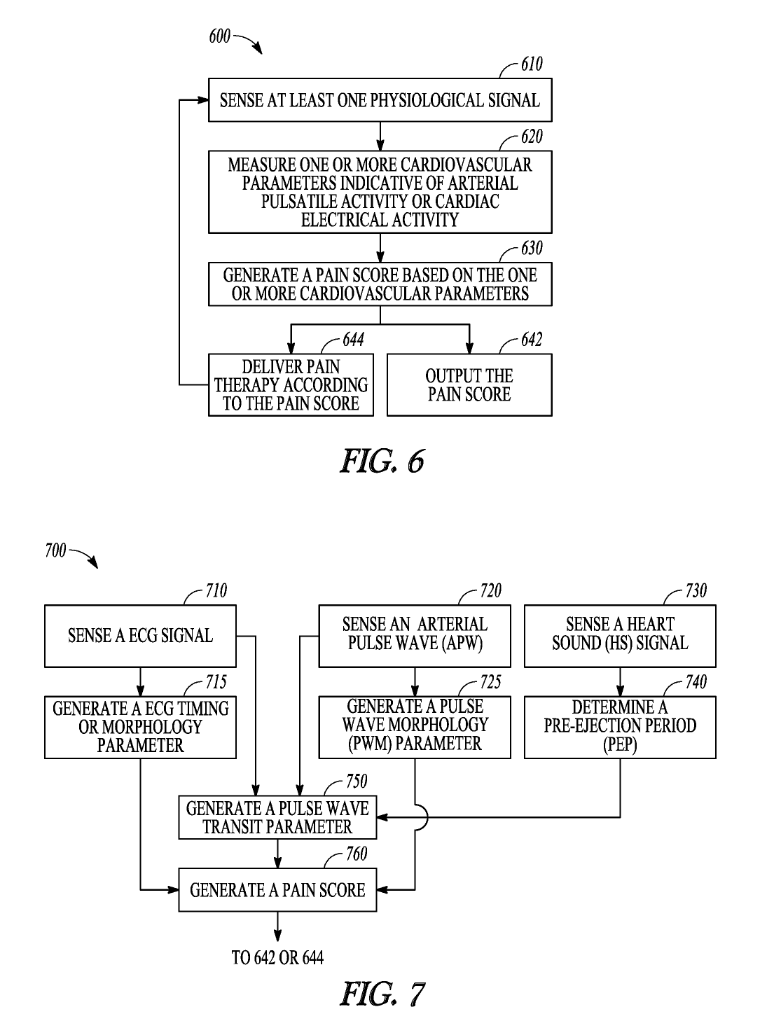

FIG. 6 illustrates, by way of example and not limitation, a method 600 for managing pain of a patient. The method 600 may be implemented in a medical system, such as the pain management system 200 or 300. In an example, at least a portion of the method 600 may be executed by a neuromodulator device (IND) such as the implantable neuromodulator 310. In an example, at least a portion of the method 600 may be executed by an external programmer or remote server-based patient management system, such as the external system 320 that are communicatively coupled to the IND. The method 600 may be used to provide neuromodulation therapy to treat chronic pain or other disorders.

The method 600 begins at step 610, where one or more physiological signals may be sensed such as via electrodes or ambulatory sensors associated with the patient. Examples of the physiological signals may include cardiac, pulmonary, or neural signals, such as, by way of example of limitation, electrocardiograph (ECG) or intracardiac electrogram, heart rate signal, heart rate variability signal, cardiovascular pressure signal, or heart sounds signal, respiratory signal, a thoracic impedance signal, or a respiratory sounds signal, or neural activity signal. The physiological signals may also include blood chemistry measurements or biomarkers that are indicative of onset, intensity, severity, duration, or different patterns of pain. In some examples, one or more functional signals may further be sensed at 610. Examples of the functional signals may include patient posture, gait, balance, physical activity signals, or signals indicating sleep or awake state, among others. Such functional signals may responsively co-variate with a pain episode. In an example, the functional signals may be sensed using accelerometer sensors. In some examples, one or more of the physiological signals may be acquired during a transition of a functional signal, such as changes in posture or when the patient goes to sleep.

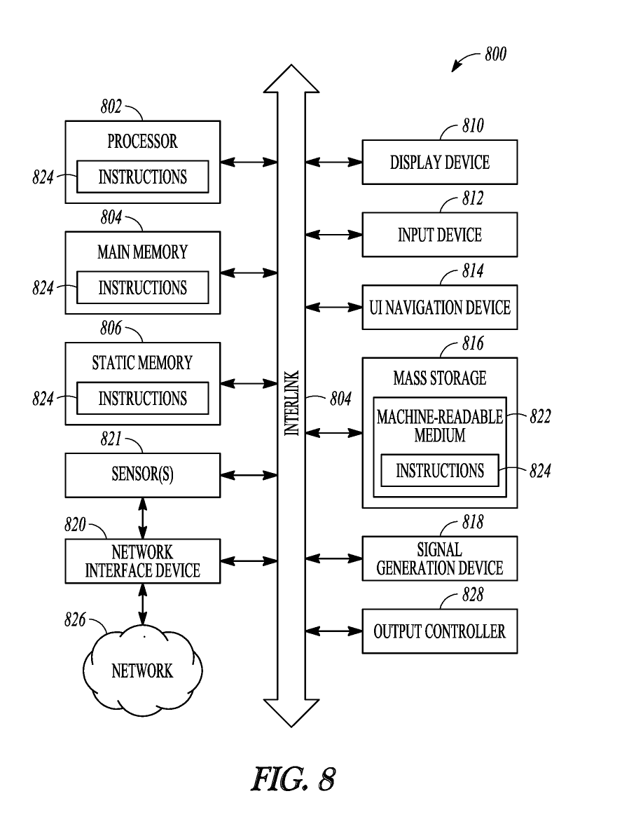

At 620, signal metrics including at least one cardiovascular parameter may be generated from the sensed physiological or functional signals. The signal metrics may include statistical parameters, morphological parameters, or temporal parameters. In an example, the signal metrics may include one or more cardiovascular parameters such as a pulse wave parameter indicative of patient arterial pulsatile activity, or an electrocardiography (ECG) parameter indicative of patient cardiac electrical activity. Examples of the pulse wave parameter may include a pulse wave transit parameter that describes conduction of the arterial pulse through a patient circulatory system, or a pulse wave morphology parameter which describes pressure distribution or blood volume dynamics when the blood propagates along the vascular system. The cardiovascular parameters or the ECG parameter may be correlated to the patient pain such as through a sympathetic and parasympathetic nervous system control, and can be used to quantify the patient pain. Examples of methods for characterizing pain using the cardiovascular parameters are discussed below, such as with reference to FIG. 7.

At 630, a pain score may be generated using the measurements of the signal metrics such as one or more cardiovascular parameters. The pain score may be represented as a numerical or categorical value that quantifies overall pain quality in the subject. In an example, a composite signal metric may be generated using a linear or nonlinear combination of the signal metrics respectively weighted by weight factors. The composite signal metric may be categorized as one of a number of degrees of pain by comparing the composite signal metric to one or more threshold values or range values, and a corresponding pain score (such as numerical values from 0 to 10) may be assigned based on the comparison.

In another example, each signal metric may be compared to a respectively specified threshold or range values and a corresponding signal metric-specific pain score may be determined. The metric-specific pain score may be determined based on a comparison of a cardiovascular parameter, such as an arterial pulse wave parameter or an ECG parameter, to a morphological template that represents individualized or population-based representative cardiovascular parameter when the patient experiences pain. A similarity measure between the cardiovascular parameter and the respective morphological template may be computed, and the metric-specific pain score may be computed based on the similarity measure. Examples of the similarity measure may include distance in a normed vector space (such as L1 norm, L2 norm or Euclidian distance, and infinite norm), correlation coefficient, mutual information, or ratio image uniformity, among others.

A composite pain score may be generated using a linear or nonlinear fusion of the signal metric-specific pain scores each weighted by their respective weight factors. Examples of the fusion algorithm may include decision trees, voting, weighted averages, or neural networks, among others. In some examples, the pain score may be computed using a subset of the signal metrics selected based on their temporal profile of pain response. Signal metrics with quick pain response (or a shorter transient state of response) may be selected to compute the pain score during a pain episode. Signal metrics with slow or delayed pain response (or a longer transient state of response before reaching a steady state) may be used to compute the pain score after an extended period following the onset of pain such as to allow the signal metrics to reach steady state of response. In some examples, patient demographic information such as patient age or gender may be used in computing the pain score. A higher pain threshold for the composite signal metric may be selected for male patients than for female patients. Additionally or alternatively, the respective weight factors may be determined based on patient demographic information. The weight factors for the signal metrics may be tuned to a lower value than the weight factors for the same signal metric in a female patient. Examples of quantizing pain using cardiovascular parameters are discussed below, such as with reference to FIG. 7.

At 642, the pain score may be output to a user or to a process, such as via the output unit 242 as illustrated in FIG. 2. The pain score, including the composite pain score and optionally together with metric-specific pain scores, may be displayed on a display screen. Other information such as physiological signals, cardiovascular parameters, or other signal metrics extracted from physiological or functional signals may also be output for display or for further processing. In some examples, alerts, alarms, emergency calls, or other forms of warnings may be generated to signal the system user about occurrence of a pain episode or aggravation of pain as indicated by the pain score. In some examples, the alert may indicate an elevated blood pressure in response to the pulse wave transit parameter satisfying a specified condition, such as the PWTT falling below a specified threshold, or the PWV exceeding a specified threshold.

The method 600 may include, at 644, an additional step of delivering a pain therapy to the patient according to the pain score. The pain therapy may include electrostimulation therapy, such as spinal cord stimulation (SCS) via electrodes electrically coupled to the electrostimulator. The SCS may be in a form of stimulation pulses that are characterized by pulse amplitude, pulse width, stimulation frequency, duration, on-off cycle, waveform, among other stimulation parameters. Other electrostimulation therapy, such as one or a combination of DBS, FES, VNS, TNS, or PNS at various locations, may be delivered for pain management. The pain therapy may additionally or alternatively include a drug therapy such as delivered by using an intrathecal drug delivery pump.

In various examples, the pain therapy (such as in the form of electrostimulation or drug therapy) may be delivered in a closed-loop fashion. Therapy parameters, such as stimulation waveform parameters, stimulation electrode combination and fractionalization, drug dosage, may be adaptively adjusted based on at least the pain score. The pain-relief effect of the delivered pain therapy may be assessed based on the signal metrics such as the cardiovascular parameters, and the therapy may be adjusted to achieve desirable pain relief. The therapy adjustment may be executed continuously, periodically at specified time, duration, or frequency, or in a commanded mode upon receiving from a system user a command or confirmation of parameter adjustment. In an example, if the pain score exceeds the pain threshold (or falls within a specified range indicating an elevated pain), then the first electrostimulation may be delivered. Conversely, if the composite pain score falls below a respective threshold value (or falls within a specified range indicating no pain or mild pain), then a second pain therapy, such as second electrostimulation may be delivered. The first and second electrostimulations may differ in at least one of the stimulation energy, pulse amplitude, pulse width, stimulation frequency, duration, on-off cycle, pulse shape or waveform, electrostimulation pattern such as electrode configuration or energy fractionalization among active electrodes, among other stimulation parameters. The method 600 may proceed at 610 to sense physiological or functional signals in response to the therapy delivered at 644. In some examples, the responses of the signal metrics to pain therapy delivered at 644 may be used to gauge composite pain score computation such as by adjusting the weight factors. In an example, weight factors may be determined and adjusted via the weight generator 322 as illustrated in FIG. 3, to be proportional to signal metric's sensitivity to pain.

FIG. 7 illustrates, by way of example and not limitation, a method 700 for quantizing pain using cardiovascular parameters. The method 700, which is an embodiment of the method 600, can be implemented in and executed by the pain management system 200 or 300 as illustrated in FIGS. 2 and 3. A composite pain score may be generated using one or more cardiovascular parameters such as generated by the cardiovascular parameter generator 400 as illustrated in FIG. 4.

The method 700 begins at 710 where an electrocardiography (ECG) signal may be sensed, such as using the sensor circuit 210 coupled to an ECG sensor. The ECG signal may be processed, and at 715 one or more of an ECG timing parameter or an ECG morphology parameter may be generated such as using the cardiovascular parameter generator 400 or the pulse wave parameter generator 520. As previously discussed with reference to FIG. 4, the ECG timing parameter may include P-P interval, P-R interval, QRS duration, Q-T interval, or ST segment duration; and the ECG morphology parameter may include amplitude of QRS, slope of R wave, or ST segment elevation, the parameters extracted from the VCG, or parameters from ECG representation in a polar coordinate system or a spherical coordinate system such as QRS complex azimuth angle, QRS complex elevation angle, T wave azimuth angle, or T wave elevation angle, among others. Changes in ECG timing or morphology parameters may indicate alterations in autonomic function such as increased sympathetic tone, which may be resulted from pain symptoms of a patient. The ECG timing or morphology parameters generated at 715 may be used for characterizing and quantifying pain.

At 720, an arterial pulse wave may be sensed, such as using the sensor circuit 210 coupled to a sensor for sensing arterial pulsatile activity. Examples of the sensor, such as the second sensor 504 in FIG. 5, may include a pressure sensor, a photoplethysmography (PPG) sensor, an impedance sensor, an accelerometer sensor, or a camera. The arterial pulse wave signal may be processed, and at 725 a pulse wave morphology (PWM) parameter may be generated, such as using the cardiovascular parameter generator 400 or the pulse wave parameter generator 520. As previously discussed with reference to FIG. 4, the PWM parameter are morphological metrics such as signal intensity (amplitude or power), change or rate of change of signal intensity at different temporal phases of the arterial pulse waveform including, for example, systolic upstroke, systolic peak, systolic decline, dicrotic notch, dicrotic runoff, and end-diastolic pressure. The PWM parameter may be correlated to cardiovascular functions such as myocardial contractility, which may be innervated by autonomic nervous system (e.g., an elevated sympathetic tone results in an increase in myocardial contractility). The PWM parameter at 725 may be used for characterizing and quantifying pain.

At 730, a heart sound signal may be sensed, such as using the heart sound sensor circuit 510. At 740, a pre-ejection period (PEP) may be measured at least based on the HS signal. In an example, the PEP is determined as a time interval between the R wave and S1 heart sound from the sensed HS signal within the same cardiac cycle.

At 750, a pulse wave transit parameter may be generated using the sensed ECG signal, the sensed arterial pulse wave, and the PEP, such as using the cardiovascular parameter generator 400 or the pulse wave parameter generator 520. The pulse wave transit parameter represent characteristic of conduction of the arterial pulse through a patient circulatory system. The pulse wave transit parameter may include a pulse wave transit time (PWTT) parameter and a pulse wave velocity (PWV) parameter. As previously discussed with reference to FIG. 4, the PWTT parameter indicates time it takes an arterial pressure waveform to propagate from one location to another location of the vascular system, and the PWV parameter represents a propagation speed of the arterial pulse wave along a length of the arterial tree, such as between two physiological events respectively detected at two different locations of the vascular system.

In an example, the pulse wave transmit parameter may be determined using a time interval between a first (S1) HS component from the sensed HS signal and an onset of the sensed arterial pulse wave onset indicating an onset of the arterial pulsatile activity. In another example, the pulse wave transit parameter may be determined based on timings of two physiological events detected at two different locations along the artery, such as wavefronts (APW1 and APW2) of the arterial pulse wave respectively detected at the two locations, and the PWTT may be determined using a time interval between APW1 and APW2. In yet another example, the pulse wave transit parameter may be determined based on an R-APW interval which is an interval between R wave on the sensed ECG signal and an onset of the sensed APW, and the pre-ejection period (PEP). The PEP represents the time period between when the ventricular contraction occurs and the semilunar valves open and blood ejection into the aorta commences. The pulse wave transit parameter may be determined as the R-APW interval less the PEP. Because the PEP reflects the propagation of blood pressure and flow within the heart, by subtracting the PEP from the R-APW interval, the resultant time period more specifically represents arterial pulse transit time through the artery from the aorta.

At 760, a pain score may be generated using one or more cardiovascular parameters generated at 715, 725, and 750. In an example, as discussed previously with reference to step 630 of the method 600, a composite signal metric may be generated using a linear or nonlinear combination of one or more of the ECG timing parameter, the ECG morphology parameter, the PWM parameter, or the pulse wave transit parameter (such as the PWTT or the PWV parameter). The composite pain score may be output to a system user or a process at 642, or to guide a pain therapy at 644.

FIG. 8 illustrates generally a block diagram of an example machine 800 upon which any one or more of the techniques (e.g., methodologies) discussed herein may perform. Portions of this description may apply to the computing framework of various portions of the LCP device, the IMD, or the external programmer.

In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810 (e.g., a raster display, vector display, holographic display, etc.), an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as a global positioning system (GPS) sensor, compass, accelerometer, or other sensor. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 816 may include a machine readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine readable media.

While the machine readable medium 822 is illustrated as a single medium, the term “machine readable medium” may include a single medium or multiple media (e.g., a centralized or distributed database, and/or associated caches and servers) configured to store the one or more instructions 824.

The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may further be transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for managing pain of a patient, the system comprising:
a sensor circuit configured to sense at least one physiological signal;
a pain analyzer circuit coupled to the sensor circuit, the pain analyzer circuit configured to:
measure, from the sensed at least one physiological signal, one or more cardiovascular parameters indicative of arterial pulsatile activity or cardiac electrical activity; and
generate a pain score based on the measured one or more cardiovascular parameters; and
an output unit configured to output the pain score to a user or a process.

2. The system of claim 1, wherein:
the sensor circuit is coupled to a first sensor configured to sense a first physiological event and a second sensor configured to sense a second physiological event, the second physiological event occurring temporally subsequent to the first physiological event; and
the one or more cardiovascular parameters include a pulse wave transit parameter indicating arterial pulse wave propagation through a patient circulatory system during a period between the first and second physiologic events.

3. The system of claim 2, wherein:
the pulse wave transit parameter includes a pulse wave transit time (PWTT) elapsed from the first physiological event to the second physiological event; and
the pain analyzer circuit is further configured to generate the pain score based on a reduction of the PWTT from a baseline PWTT.

4. The system of claim 3, further comprising a third sensor configured to sense a heart sound (HS) signal, wherein:
the first sensor is further configured to sense an R wave in an electrocardiogram (ECG) signal;
the second sensor is further configured to sense an arterial pulse wave (APW) signal; and
the pain analyzer circuit is further configured to:
determine a pre-ejection period (PEP) based on at least the sensed HS signal;
determine a R-APW time interval between the sensed R wave and an APW onset indicating an onset of the arterial pulsatile activity; and
determine the PWTT based on a difference between the R-APW time interval and the PEP.

5. The system of claim 2, wherein:
the first sensor is further configured to sense a heart sound (HS) signal;
the second sensor is further configured to sense an arterial pulse wave (APW) signal; and
the pain analyzer circuit is further configured to determine the PWTT based on a time interval between (1) a first (S1) HS component from the sensed HS signal and (2) an APW onset indicating an onset of the arterial pulsatile activity.

6. The system of claim 2, wherein:
the pulse wave transit parameter includes a pulse wave velocity (PWV) indicative of a propagation speed of the arterial pulse wave between the first and second physiological events; and
the pain analyzer circuit is further configured to generate the pain score based on an increase of the PWV from a baseline PWV.

7. The system of claim 2, wherein at least one of the first sensor or the second sensor includes at least one of:
a pressure sensor;
a photoplethysmography (PPG) sensor;
an impedance sensor;
an accelerometer sensor; or
a camera configured to capture an image indicative of arterial blood flow.

8. The system of claim 1, wherein the pain analyzer circuit is further configured to measure the one or more cardiovascular parameters including a pulse wave morphological parameter.

9. The system of claim 1, wherein the pain analyzer circuit is further configured to measure the one or more cardiovascular parameters including an electrocardiography (ECG) timing parameter or an ECG morphological parameter.

10. The system of claim 1, further comprising:
an electrostimulator configured to generate electrostimulation energy to treat pain; and
a controller circuit coupled to the pain analyzer circuit and the electrostimulator, the controller circuit further configured to control the electrostimulator to deliver a pain therapy and to control the electrostimulation energy generated by the electrostimulator according to the pain score.

11. The system of claim 10, wherein the controller circuit is further configured to deliver first electrostimulation to the patient in response to the pain score exceeding a threshold value, and to deliver second electrostimulation to the patient in response to the pain score falling below the threshold value;
wherein the first electrostimulation differs from the second electrostimulation with respect to at least one of electrostimulation energy, an electrostimulation pulse shape, or an electrostimulation pattern.

12. The system of claim 10, further comprising an implantable neuromodulator device (IND) that includes one or more of the sensor circuit, the pain analyzer circuit, or the electrostimulator.

13. A method for managing pain of a patient using an implantable neuromodulator device (IND), the method comprising:
sensing at least one physiological signal from the patient via a sensor circuit;
measuring, from the sensed at least one physiological signal, one or more cardiovascular parameters indicative of arterial pulsatile activity or cardiac electrical activity;
generating a pain score based on the measured one or more cardiovascular parameters; and
outputting the pain score to a user or a process.

14. The method of claim 13, further comprising delivering a pain therapy via the IND, the pain therapy including electrostimulation energy determined according to the pain score.

15. The method of claim 13, wherein:
the sensed at least one physiological signal includes a first physiological event and a second physiological event that occurs temporally subsequent to the first physiological event; and
the one or more cardiovascular parameters include a pulse wave transit parameter indicating an arterial pulse wave propagation through a patient circulatory system during a period between the first and second physiologic events.

16. The method of claim 15, wherein:
the pulse wave transit parameter includes a pulse wave transit time (PWTT) elapsed from the first physiological event to the second physiological event; and
the pain score is generated based on a reduction of the PWTT from a baseline PWTT.

17. The method of claim 16, further comprising:
sensing a heart sound (HS) signals using a HS sensor;
determining a pre-ejection period (PEP) based on at least the sensed HS signal; and
measuring an R-APW time interval between an R wave of an electrocardiogram (ECG) and an arterial pulse wave (APW) onset indicating an onset of the arterial pulsatile activity; and
wherein the PWTT is determined as a difference between the R-APW time interval and the PEP.

18. The method of claim 15, wherein:
the pulse wave transit parameter includes a pulse wave velocity (PWV) indicative of a propagation speed of the arterial pulse wave between the first and second physiological events; and
the pain score is generated based on an increase of the PWV from a baseline PWV.

19. The method of claim 13, wherein the one or more cardiovascular parameters include a pulse wave morphological parameter.

20. The method of claim 13, wherein the one or more cardiovascular parameters include an electrocardiography (ECG) timing parameter or an ECG morphological parameter.
